(19) United States
(12) Patent Application Publication
(10) Pub. No.: US 2005/0020928 A1
(43) Pub. Date: Jan. 27, 2005

(54) Non-invasive detection of endothelial dysfunction by blood flow measurement in opposed limbs

(76) Inventor: Andre Arsenault, Longueuil (CA)

Correspondence Address: OGILVY RENAULT, 1981 MCGILL COLLEGE AVENUE, SUITE 1600, MONTREAL, QC, H3A2Y3, CA

(21) Appl. No.: 10/871,538

(22) Filed: Jun. 21, 2004

Related U.S. Application Data
(63) Continuation of application No. PCT/CA01/01834, filed on Dec. 19, 2001.

(51) Int. Cl. A61B 5/02

(52) U.S. Cl. 600/504; 600/431; 600/549; 600/310

(57) ABSTRACT

Endothelial dysfunction is a known indicator of coronary artery disease. Endothelial dysfunction is detected by measuring presence of a marker in arteries following the release of blood flow into the limb after a period of blockage of blood flow into the limb. The blood flow is measured in a pair of laterally opposed limbs, such as the patient's forearms, and the marker presence is compared between both limbs. One efficient marker is a tracer containing a radionuclide and the non-invasive measurement of the radionuclide is carried out by gamma ray detection.

### CROSS-REFERENCE TO RELATED APPLICATIONS

This application is a continuation of PCT International patent application serial number PCT/CA01/01834, filed Dec. 19, 2001, and now pending, and claims priority of U.S. patent application Ser. No. 09/603,554 filed Jun. 26, 2000, now U.S. Pat. No. 6,445,945, the specification of which is hereby incorporated by reference.

### FIELD OF THE INVENTION

The present invention relates to the diagnosis of endothelial dysfunction, particularly in humans. The non-invasive technique involves blocking blood flow in a limb to stimulate endothelial function and then releasing the blood flow block to observe blood flow related changes which are indicative of endothelial dysfunction. More specifically, the present invention relates to a method and apparatus for conducting such observations.

### BACKGROUND OF THE INVENTION

In recent years, the connection between endothelial dysfunction and the risk of arteriosclerosis has been studied and established (see the article by Celermajer et al., “Non-Invasive Detection of Endothelial Dysfunction in Children and Adults at Risk of Arteriosclerosis”, Lancet, 1992, Vol. 340, pages 1111 to 1115, and the article by Schächinger et al., “Prognostic Impact of Coronary Vasodilator Dysfunction on Adverse Long-Term Outcome of Coronary Heart Disease”, published in Circulation, 2000, Vol. 101, pages R1 to R8).

The most popular technique for measuring blood flow for the purposes of endothelial dysfunction in children and adults is the use of Doppler ultrasound which is able to obtain a measurement of blood flow in an artery of a patient non-invasively. As can be appreciated, this requires placing an ultrasound transceiver directly on top of an artery and the measurement accuracy is dependent on proper positioning of the ultrasound equipment with respect to the artery. The paper authored by Todd J. Anderson entitled “Assessment and Treatment of Endothelial Dysfunction in Humans” provides a review of known techniques for assessment of endothelial function in humans. These techniques include intracoronary studies, positron emission tomography, impedance plethysmography, brachial ultrasound (also known as Doppler ultrasound) and venous studies. This article was published in Vol. 34, Issue 3, (September 1999), pages 631-638 of JACC. Moreover, the interest of a combined method of assessing endothelial dysfunction and myocardial perfusion was stated by Herrmann in a recent review article (J Nucl Cardiol Vol. 8, Issue 2 (March/April 2001) page 204: “Thus scintigraphy in combination with endothelial testing strategies may be used to redefine the pathophysiologic role and prognostic significance of endothelial dysfunction in patients with epicardial disease, microvascular disease, or both.”

The fact that endothelial dysfunction is an indicator of the risk of events (infarction, unstable angina) in coronary artery disease (CAD) makes the detection of endothelial dysfunction of great value in the diagnosis and treatment of the target groups within the general population. People can be at risk of heart disease and CAD as a result of family history, diabetes, obesity, hypertension, as well as environmental factors (such as the presence of first-hand or second-hand smoke), diet and age. The ability to provide for an efficient non-invasive test for the risk of stratification of arteriosclerosis would be a valuable tool to determine whether more complex tests are needed to determine the presence of CAD or whether such further tests can be dismissed as unnecessary. Full coronary angiography consumes time on equipment costing in the range of $500,000 to $1,000,000, and require significant operator training and analysis by a skilled specialist. The cost savings to avoiding expensive tests is significant.

The ability to test endothelial dysfunction as an indicator of the state of CAD is also useful for the purposes of monitoring a patient's response to medical treatment, i.e. drugs, diet, exercise, stress management, or a combination thereof. It would also be useful to monitor the residual persistence of risk after revascularization procedures such as coronary bypass and coronary angioplasty.

It would therefore be desirable to provide for a test which would be reliable, easy to carry out, inexpensive and non-invasive for the purposes of determining endothelial dysfunction in humans.

### SUMMARY OF THE INVENTION

It is an object of the present invention to provide an accurate method and apparatus for detecting endothelial dysfunction in humans which overcomes the drawbacks associated with prior art methods, for example by not measuring blood flow velocity and instead by measuring concentration or presence (or changes therein) of detectable substances within a limb non-invasively.

It is an object of the present invention to provide an accurate method and apparatus for detecting endothelial dysfunction in humans, which involves a comparatively low cost.

It is an object of the present invention to provide an accurate method and apparatus for detecting endothelial dysfunction in humans which is easy to carry out.

It is an object of the present invention to provide an accurate method and apparatus for detecting endothelial dysfunction in humans which involves a non-invasive approach.

It is an object of the present invention to provide an accurate method and apparatus for detecting endothelial dysfunction in humans to provide an accuracy sufficient for a screening-level quality of results in order to determine whether patients should proceed to more substantial medical tests or observations with respect to heart or cardiovascular disease.

The present invention relates to a method and apparatus for detecting ingress of a substance into the limb following the release of the transient blockage of blood flow.

In one embodiment, the invention involves injecting a tracer substance and imaging or otherwise detecting the tracer ingress into the limb following the release of the blood flow block. In some embodiments, the trace substance is a radiation emitter, and in others, a contrast agent.

In another embodiment, the invention involves measuring by suitably accurate means a physical property of a metabolic or other biochemical product circulating in the limb following the release of the blood flow block. Either the appearance rate of a depleted substance like O₂ or the disappearance (depletion) of an accumulated product like CO₂ may be detected. Suitable techniques may include gas emissions, e.g. O₂ or CO₂, across the skin surface within a cell placed on the skin surface, optical techniques, such as spectral analyzers or optical transmission/diffusion detectors, such as the visible-reflectance hyperspectral analysis as described recently by Zuzak (Circulation 2001; 104:2905-2910), and EPR/NMR based techniques, such as detection of deoxy-haemoglobin that contrary to oxy-haemoglobin has paramagnetic properties (see David D. Stark “Magnetic Resonance Imaging.” 2nd Edition, Mosby 1992 p.721).

According to the invention, suitable measurements may be only differences in metabolic product or tracer product-induced property levels before blocking or occlusion and after. According to the invention, suitable measurements may also be only differences in metabolic product or tracer product-induced property levels between limbs. The use of differential measurements may be exploited to avoid problems associated with calibration to an absolute scale, and processing of the signals measured to provide valuable results may be achieved according to the invention. Also, the invention provides using a rate of change of the measured parameter shortly after the occlusion or blockage is released as a primary factor in determining endothelial dysfunction. Preferably, in the case of the use of a tracer, the rate of both the blocked limb and the contra-lateral (control) limb is measured.

According to a first broad aspect of the invention, there is provided a method for diagnosing endothelial dysfunction by measuring tracer presence in arteries following the release of blood flow into the limb after a period of blockage of blood flow into the limb. According to one aspect of the invention, such blood flow is measured in a pair of laterally opposed limbs, preferably the forearms, and the tracer presence is compared between both limbs. The tracer is also preferably a radionuclide and the non-invasive measurement of the radionuclide is carried out by gamma ray detection.

According to another aspect of the invention, there is provided a device for guiding and mounting a person's forearms over a detector measuring tracer presence within a region of interest in the forearm. In one embodiment, the guide is used for holding, in a predetermined position, a person's forearm over a conventional 2-D gamma camera.

According to another embodiment, the guide is used to hold a person's forearm in a fixed position with respect to a detector measuring the tracer presence in which the detector is located and maintained over a region of interest and is not required to form a two-dimensional image of the region of interest.

According to yet another embodiment of the invention, a detector for detecting radiation emitted from a radionuclide is provided within a band surrounding a person's limb for detection of radiation.

It will be understood that several embodiments of the invention involve measuring tracer presence in two laterally opposed limbs of a person in which steps are taken to ensure that the sensitivity of measurement between both limbs is the same.

It will also be understood that several embodiments of the present invention involve the injection of a bolus of a radioactive tracer in a vein of a person. Preferably, the dosage strength of the radioactive tracer is measured by a detector prior to injection in order to obtain a reference calibration point. Preferably, the detector used for calibration is the detector used for measuring the tracer in both limbs.

It will be appreciated that flow change measurement is a parameter that can be used without knowing or measuring flow per se. The most sensitive parameter would thus be the peak flow rate per unit of time. More precisely, any detectable molecule or marker or physical characteristic that will change in proportion to the flow rate and fast enough to produce at least one valid observation per second so that the change in flow is not lost in a too large integration constant over time.

### BRIEF DESCRIPTION OF THE DRAWINGS

The present invention will be better understood by way of the following detailed description of preferred embodiments of the invention with reference to the appended drawings in which:

FIG. 1 is flow chart of the method according to the preferred embodiments;

FIG. 2 is a graph obtained from clinical studies of a patient exhibiting normal hyperemia showing count rate as a function of time, the left-hand graph illustrating an expanded view of the first few seconds after bolus injection and the right-hand graph illustrating the count rate over time extending into a steady state region after several minutes;

FIG. 3 is a graph similar to FIG. 2 for a patient exhibiting abnormal hyperemia, i.e. endothelial dysfunction;

FIG. 4 illustrates a two-dimensional image obtained using a conventional two-dimensional gamma camera of a pair of forearms placed over a gamma camera surface showing the progression of image acquisition over the first 8 seconds in which the imaging of the radioactive isotope flowing in the pair of arteries in each forearm can be clearly seen up until the point that the radioactive isotope penetrates into the tissue of each forearm (the illustration of FIG. 4 corresponds to normal hyperemia);

FIG. 5 is a plan view of a forearm support guide for mounting to the surface of a conventional gamma camera according to the first preferred embodiment;

FIG. 6 is a side view of the device according to FIG. 5;

FIG. 7 is a side view of the apparatus according to the second preferred embodiment in which a single scintillation detector is located at the region of interest for a first forearm;

FIG. 8 is a lateral end view of the apparatus according to the third preferred embodiment in which a pair of detectors, as per FIG. 7, are rotatably mounted to a forearm support surface wherein the detectors can be rotated to face a support for holding the radioactive bolus between the two detectors equidistantly therebetween;

FIG. 9 is a side view of the apparatus according to the fourth preferred embodiment in which a pliable radiation detector is wrapped around the limb; and

FIG. 10 is a detailed view of the pliable radiation detector according to the fifth preferred embodiment in which scintillation fibers extending circumferentially on the inside of a pliable casing are connected to optical fibers of an optical fiber bundle connected to a light detector or photomultiplier tube (PMT).

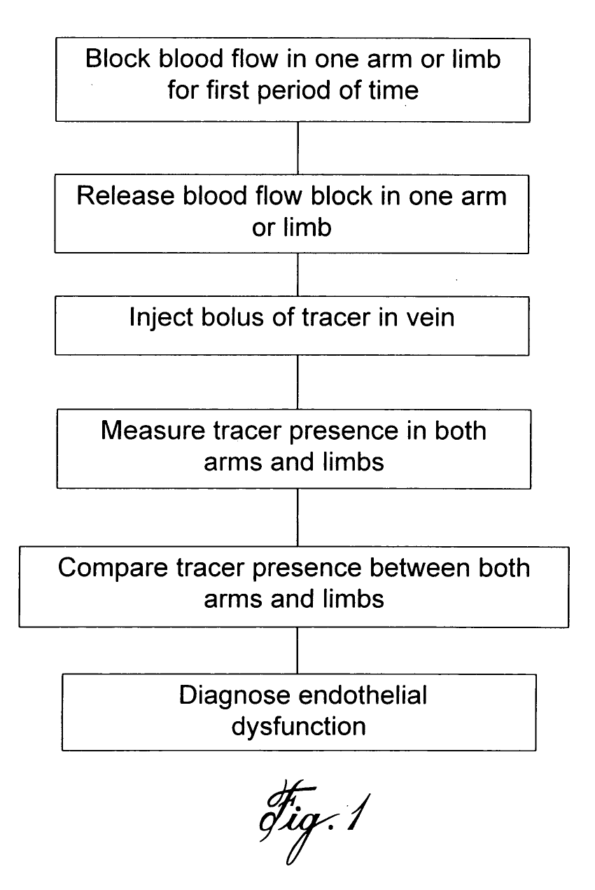

### DETAILED DESCRIPTION OF THE PREFERRED EMBODIMENTS

Applicants have tested in a clinical environment the measurement of the presence of a radioactive tracer in two forearms of a patient using a conventional gamma ray or scintillation camera. Such a camera is able to provide an image of the increasing presence of a radioactive isotope entering the arms following the injection of a bolus of the tracer in a vein. In the clinical experiments conducted, the bolus of tracer was injected in a patient's upper arm in a vein, which would bring the bolus of tracer to the heart for even distribution to both the left arm and the right arm, with a slight delay for the left arm. For the purposes of testing endothelial dysfunction, blood flow is blocked for a period of time, such as a few minutes to several minutes. The blockage of blood flow in the one arm followed by the subsequent release of the blood flow blockage would lead to a substantially increased blood flow in the arm previously blocked which bodily function is referred to as normal hyperemia. This function is possible when there is no endothelial dysfunction. It is preferred that the injection of the bolus be administered to the unblocked arm.

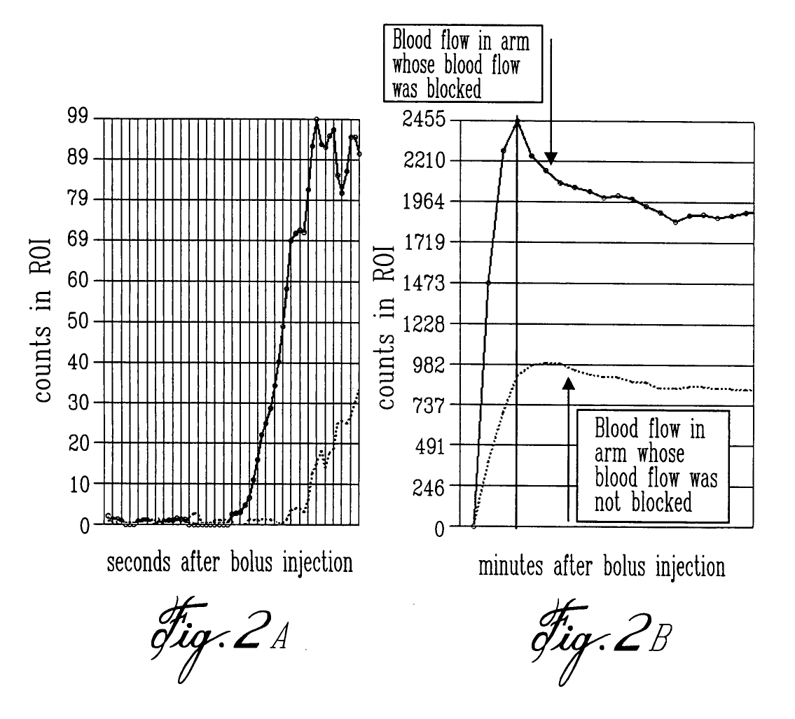

In the case of FIG. 2, the healthy patient exhibits a significant rapid increase of blood flow in the arm which was previously blocked.

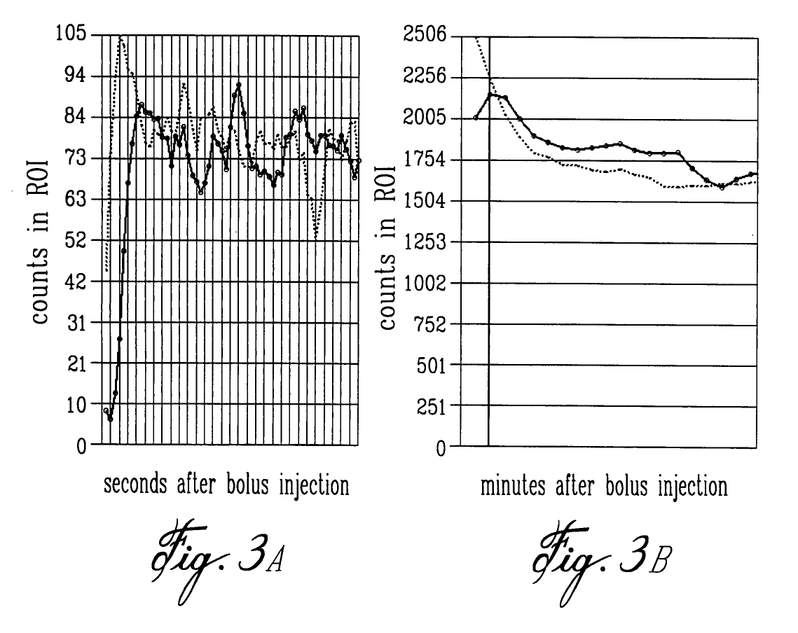

As illustrated in FIG. 4, the right-hand arm shows presence of the radioactive tracer at a much greater rate of increase in comparison to the left forearm. The number of counts illustrated in FIGS. 2 and 3 can be measured by integrating the counts found in any particular area within the two-dimensional image acquired using the conventional scintillation camera. The choice of area over which the number of counts is to be integrated is to be chosen taking into consideration a number of factors. Applicants prefer to choose a region that is not too close to the elbow and not too close to the wrist. While the larger the area chosen, the greater number of counts obtained, it may be desirable to choose a restricted area such as an area corresponding to each artery. Applicants have found that choosing a medial area of approximately the width of the forearm and approximately mid-distance between the elbow and the wrist provides satisfactory results. In the data collected in FIG. 4, a patient placed his forearms directly on the scintillation camera screen without the use of a guiding device. Care was therefore taken to select the region of the whole image from each forearm for comparing the tracer presence growth in each of the forearms. It is also possible to draw multiple smaller adjacent areas of interest extending over the arms and juxtaposed all the way down to the fingers: the areas closer to the elbow reflecting flow trough larger arteries of the forearm whereas the distal ones monitor reactive hyperemia in the much smaller arteries of the fingers. This might become important since endothelial dysfunction appears to be non-homogeneous as a function of the size of arteries.

As illustrated in FIG. 3, it is clear that in the case of abnormal hyperemia, i.e. endothelial dysfunction, the increase in presence of a tracer in both forearms is substantially the same. The significant difference between normal and abnormal hyperemia allows for a clear diagnosis. In the preferred embodiments, this diagnosis is to be made using the data acquired over time, as illustrated in FIGS. 2 and 3, using a radiation detector capable of accurately resolving the count rate in the region of interest in order to show the shape of the rapid tracer presence growth in the respective forearms. While less desirable, it would be possible within the scope of the present invention to use a tracer presence detector having a much slower response which would be useful in measuring the steady state value reached after a few minutes. Alternatively, it is also within the scope of the present invention to use the measurement of tracer presence in a single limb and to derive sufficient information from such measurement to determine whether normal or abnormal hyperemia and, therefore, endothelial dysfunction occurred in the patient.

It will be appreciated that while detection using a single limb is possible, the advantages of measuring tracer presence in both limbs typically will greatly outweigh any disadvantage in needing to provide more equipment to measure tracer presence in both limbs.

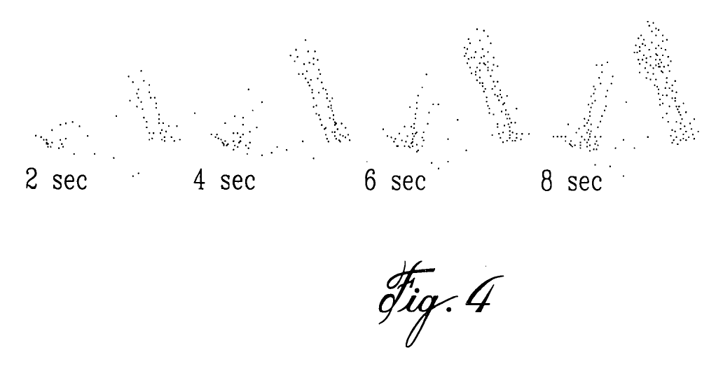

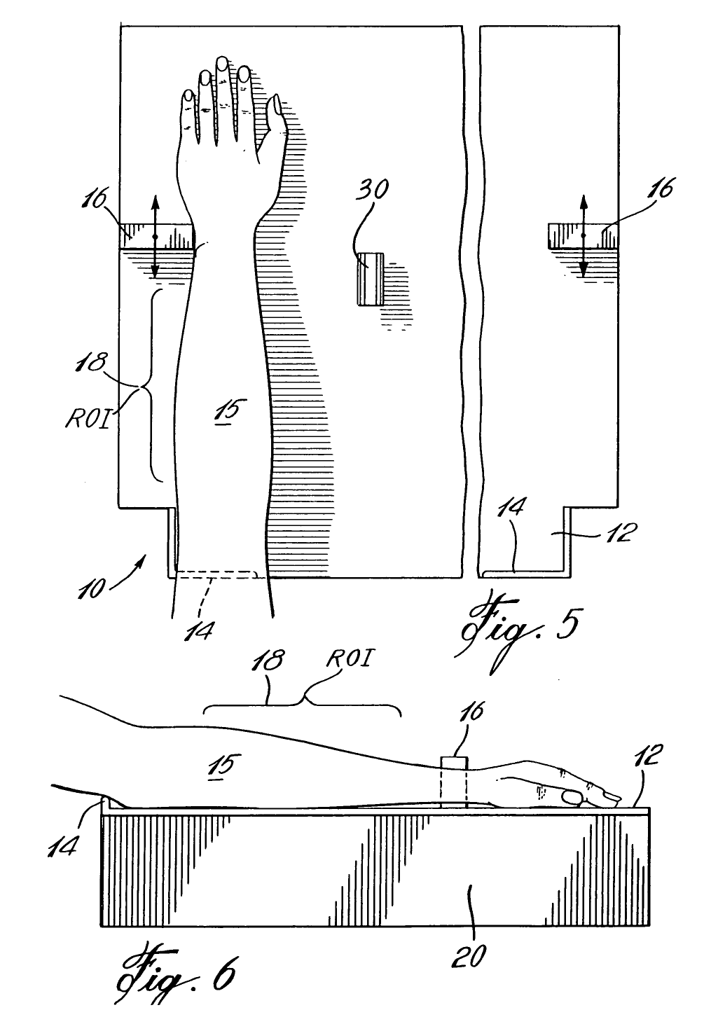

According to the first embodiment illustrated in FIGS. 5 and 6, the apparatus according to the invention 10 comprises a radiotransparent plate 12 able to be fitted over a conventional gamma camera arranged to be level and facing upwards. In order to mount the apparatus 10 to the camera 20, fasteners may be used, or the outer edges of the plate 12 may extend over and downwardly at the sides to be fixed in position while resting on the camera window. Left and right bottom corners 14 have edges for supporting the patient's elbows when pressure is exerted towards the patient and outwardly against the supports 14. To ensure that the patient's arms are in a fixed position, a slidable ulnar support 16 is placed at or just before the wrist. The purpose of choosing the support points in the embodiment of FIG. 5 is to choose locations where the patient can contact the guide device 10 with the bone substantially contacting the guide device, rather than softer tissues such as muscle. In this way, a change in patient pressure against the guide device 10 will not result in a change in forearm position. The forearm 15 includes a region of interest 18 (ROI) that is substantially a middle portion between the elbow and the wrist. In the preferred embodiment, the patient places his or her hands, palms down, on the surface 12 and as illustrated in FIG. 6, the gamma camera 20 is positioned underneath.

It will be noted that the patient's forearms are preferably positioned such that they are extended, i.e. the elbow is bent minimally, in order to reduce any obstruction in the blood flow due to compression at the elbow joint. The patient's forearms are preferably positioned ergonomically on the surface of the camera 20. In the preferred embodiment, the camera is positioned to face upward at a desired height so that the patient may sit on a chair with his or her arms extended and have his or her palms rest comfortably on the camera surface. With control, a patient may keep his forearms in a fixed position on the camera surface without abutment supports 14 and 16. Alternatively, a resilient cover, such as foam material, could be provided and placed over the forearms to help the patient keep his or her forearms in a steady and fixed position on the surface 12. Such a cover could be hinged to the surface 12 and be locked in a covering position for the duration of the test.

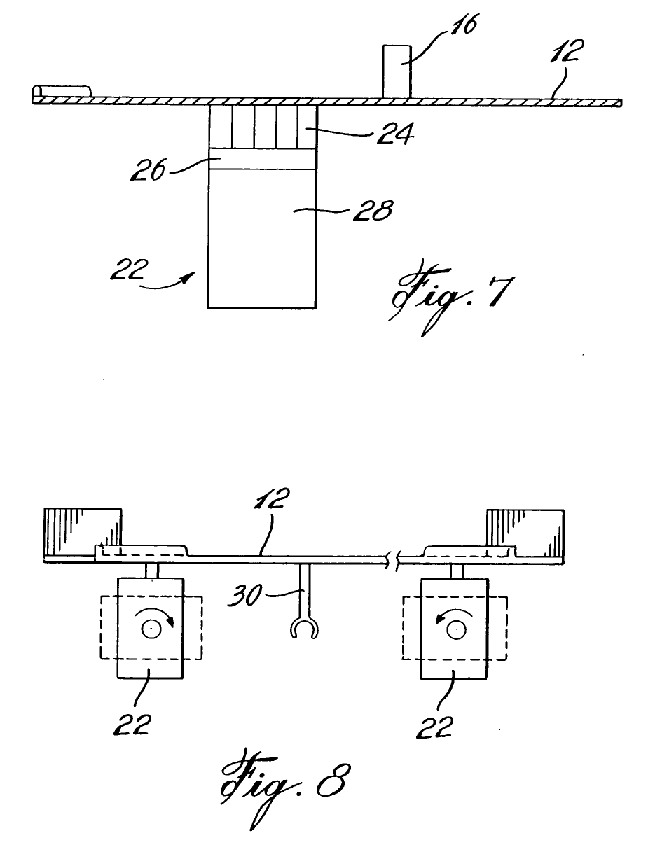

As in the embodiment of FIG. 8, the embodiment of FIGS. 5 and 6 may provide for calibration of the bolus to be used. The bolus may simply be placed in a designated region on the surface 12, as provided for by markings or by a holding device 30, e.g. similar to the holding device 30 illustrated in FIG. 8. Since camera 20 may have variation in sensitivity as a function of position, it is important to fix the position of the bolus on the surface 12 for calibration purposes.

Different configurations of abutment supports 14 and 16 can be provided. For example, finger posts, i.e. vertical posts received in the crotch between fingers, may be used to position the hand, while an elbow or lateral forearm abutment can then be used for positioning the forearm. It may also be desirable to position the forearms resting on the ulnar bone and to position the hand using a vertical grip post. It will be appreciated that the positioning devices should not interfere with blood flow and the reactive hyperemia especially in the finger areas.

In the embodiment of FIG. 7, the gamma camera 20 is replaced by a single gamma ray detector 22 consisting of a coarse (i.e. large aperture) collimator 24, a scintillation detector material 26, such as a thallium-doped sodium iodide crystal or the like 26, and a photomultiplier tube 28. Collimator 24 is typically made of lead, although steel or any suitable dense metal may be used. Commercially available probes used to measure thyroid uptake could easily be used in pairs and adapted to such measurement. The use of shielding and collimation is not as important to the present invention as in the field of nuclear imaging. However, the detector should be prevented from receiving counts from the syringe during injection of the bolus by using proper shielding techniques. Photomultiplier tubes 28 are well known in the art. In the embodiment of FIG. 7, the radiation detector 22 is located in a fixed position with respect to support surface 12 in an area which would be located at the average region of interest for a person's forearm. The position of the radiation detector 22 may also be made to slide linearly in a direction extending between the elbow stop 14 and the ulnar support 16 in order to accommodate patients of different size forearms and/or to provide for an adjustment in the position of the region of interest. To ensure that the region of interest at which radiation is detected is the same for both forearms, detector 22 in the case that it is mobile, is adjusted on one side to be in the same relative position as its complementary detector on the other side.

Although a palms down configuration and an upwardly facing detector is preferred, it may also be desirable to provide a positioning guide for a palms up or palms sideways configuration, either with the hand extended (karate chop) or closed (fist or handle grip). When supporting the forearm on the ulnar bone (palm sideways), it may also be desirable to arrange a pair of horizontally facing detectors on opposite sides of the same forearm.

In the embodiment of FIG. 8, the radiation detectors 22 are provided to be pivotable from a position in which they face the region of interest 18 of the forearm to a position in which they face each other in order to be calibrated using the bolus of radioactive tracer which is to be injected into the patient. The bolus is placed in a holder 30 provided under the surface 12 at a position midway between the two detectors 22. The calibration period may be from a few seconds to over a minute to establish an estimate of the radioactive strength of the bolus to be used. This calibration allows, at the same time, the response or sensitivity of each radiation detector to be checked and for the strength of the radioactive bolus to be measured to provide an important reference point for the subsequent measurements and diagnosis. To avoid saturation due to high count rates, the distance from the source can be increased or the syringe shielded with appropriate adjustments of counts for attenuation correction.

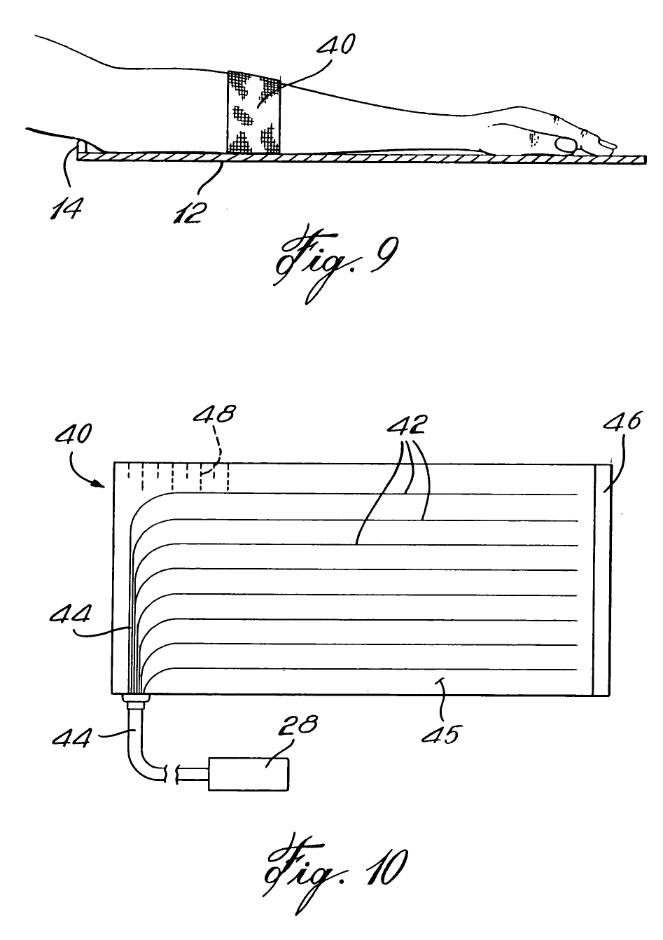

In the embodiment of FIG. 9, the radiation detector is provided in a manner which surrounds the limb, such as a leg or a forearm 18. In this embodiment, the radiation detector comprises a plurality of scintillation fibers, as are known in the art, which are arranged within a pliable support 45 to extend circumferentially around the limb without any appreciable pressure which could affect blood flow in the limb. The pliable casing 45 may provide shielding such as a lead blanket or the like. The pliable casing 45 is fastened using a strip of hook and loop type fastener 46 which mates with the complementary material provided on the underside of the pliable casing 45 as illustrated in FIG. 10. To ensure that the position and arrangement of the detector 40 is the same for each limb, it is preferable to provide scale markings or indicia 48 on the outside of the casing to confirm that the strip 46 is wrapped around to the same position on the outside of the pliable casing 45 on each forearm or leg. In keeping with the objective that the casing does not exert any pressure on the limb which could adversely affect blood flow, the casing may be wrapped around the limb and fastened using the fastener 46 as marked by the indicia while being somewhat loose on the limb.

Scintillation light from the fibers 42 is communicated to optical fibers 44 of a bundle which is fed into a common light detector or photomultiplier tube 28. While the detector of FIG. 10 is illustrated as comprising a number of discrete fibers 42, it may alternatively be possible to loop a single fiber 42 in a suitable arrangement, or to use a sufficiently thin film of a plastic scintillator so as to provide a scintillator sheet which is pliable around the limb. The position of the detector 40 with respect to the elbow stop 14 is also a parameter to be controlled during measurement, and scale markings or indicia on the surface of the support plate 12 or the use of a measuring tape may be useful for such purposes.

As an alternative to a soft pliable casing wrapped around a limb, it would be also possible to provide a rigid arcuate casing containing detector material, such as fibers 42. Such an arcuate casing may form a rigid bracelet or a semi-cylindrical member fitting over a limb supported on a surface. In the case of a semi-cylindrical member, the member may be hinged to a support surface. In the case of detecting a radioactive tracer in a person's forearms, the semi-cylindrical casing can be hinged to a support surface as in the embodiment of FIG. 5 or 7 which includes positioning guides for the forearm.

While the preferred embodiments disclose the use of a radioactive tracer for the purposes of measuring blood flow, tracers may also be used to measure blood flow during MRI detection and to enhance detection using conventional techniques such as impedance plethysmography and brachial ultrasound.

It will be appreciated that detectors may be arranged at a variety of different positions and orientations with respect to a limb in a manner suitable to obtain a sufficiently reliable diagnosis of endothelial dysfunction.

In accordance with another embodiment, changes in metabolic activity in the occluded arm can be detected through the measurement of either the disappearance rate of an accumulated biochemical product, like CO₂, or the appearance rate of a depleted substance like O₂ during the occlusion period. The detection system may also be able to monitor the concentration in absolute or relative terms of metabolic products that are either flowing in, like oxygen or are being flushed away like CO₂ using commercially available devices, such as the TCO₂M™ Transcutaneous Monitor device manufactured by Novametrix Medical Systems Inc. A miniaturized gas-carried sampling device can also capture trace amounts of diffusible molecules through the skin barrier and can be hooked to a chromatographic/spectrometric device for separation and quantification of such diffusible metabolic marker.

In a preferred embodiment, one arm is occluded to be 50 mm Hg above systolic blood pressure for a period of 5 minutes. This pressure measurement can be reliably conducted by a nurse or by using an automatic blood pressure measurement device comprising a logical unit to run the sequences of inflations. For example, a cycle comprises a first inflation done to monitor the actual resting blood pressure and record the systolic component, a five minute delay for recovery follows, and then a second inflation cycle detects the target pressure to be maintained as 50 mm Hg above rest systolic blood pressure. A monitoring device records the actual inflation pressures during the whole inflation period of 5 minutes to ensure that the target “blocking” was maintained. The pressure data is stored in a database. A standard blood pressure monitor may be used for the present embodiment along with an interface with a logical unit to implement the recording and control an inflation unit to reach the target pressure and maintain it for 5 minutes. The logical unit also controls a deflating valve that enables a rapid release of pressure.

The logical unit also has a printing capability to create and maintain an original hard copy of the procedure. The logical unit detects and records a baseline level of the target tracer or molecule before the inflation cycle during the five minutes recovery period. A small dose of the tracer might be injected to properly “calibrate” the limbs of the subject. This allows for the detection of any systematic difference between the limbs and insures the stability of the detected signal over time. The logical unit detects a first level of the substance at a time of the releasing of the blood flow and detects at least one second level of said substance after the releasing. The first level and the at least one second level are used to calculate a parameter indicative of endothelial health or dysfunction. In the preferred embodiment, the second level is detected at a plurality of predetermined points in time following the releasing, and a maximum rate of change in the substance detected is determined by the logical unit from the series of second level recorded values. Also, a base level of the substance prior to blocking is recorded for comparison with a “steady state” value of the second level values taken within a few minutes of the release of blood flow. As mentioned above, a higher than the base level steady state value is a sign of endothelial health, whereas a lower steady state value is a sign of endothelial dysfunction. The maximum rate of change in the presence of the detected substance can also be used directly as an indicator of endothelial dysfunction. This indicator can be advantageously combined with the steady state to base level comparison to confirm endothelial dysfunction.

In accordance with a further embodiment, changes in metabolic activity in the occluded arm can be detected through the measurement of the physical characteristics in the arms during occlusion and after release of the occlusion. While temperature alone may provide sufficient data, a combination of temperature and color may be more robust. This measurement can be done using a thermocouple and/or a color-sensing device. Optical sensing means, such as an oximeter, may also be used with efficiency without requiring a temperature measurement.

In accordance with yet a further embodiment, changes in metabolic activity in the occluded arm can be detected through the measurement of reduced hemoglobin which, contrary to oxy-haemoglobin, possesses paramagnetic properties and can be detected and measured using proper MRI devices.

The present invention has been described above with reference to a number of specific preferred embodiments. It will be appreciated that many other embodiments are contemplated within the scope of the present invention as defined in the appended claims.

1. A method of obtaining data for the diagnosis of endothelial dysfunction, the method comprising:
blocking blood flow in one limb for a first period of time;
releasing the block of blood flow in said one limb;
measuring a change in the presence of a substance in said one limb as a result of a return of blood flow in said one limb; and
determining from said change data indicative of endothelial dysfunction.

2. The method as claimed in claim 1, further comprising a step of injecting a bolus of a tracer in a vein such that said bolus is conducted to the heart and evenly distributed to said one limb and an opposed limb via arteries substantially simultaneously with said releasing, wherein said substance comprises said tracer.

3. The method as claimed in claim 2, wherein said step of measuring tracer presence comprises measuring tracer presence in both said limbs, said step of determining comprising comparing tracer presence between both said limbs.

4. The method as claimed in claim 3, wherein said tracer comprises a radioactive tracer, said step of measuring comprises measuring radiation emitted from a region of interest in said limbs.

5. The method as claimed in claim 2 wherein said tracer presence is measured and recorded as a function of time at a plurality of points in time.

6. The method as claimed in claim 4, wherein said step of measuring comprises adjusting a position of said limbs with respect to a radiation detector so as to detect radiation with a substantially equal sensitivity for each of said limbs.

7. The method as claimed in claim 6, wherein said limbs comprise arms and said region of interest is a forearm.

8. The method as claimed in claim 7, wherein said forearms are placed palms down on a substantially flat surface in order to detect said region of interest of each one of said forearms.

9. The method as claimed in claim 6, wherein diagnosis of endothelial dysfunction is determined from a steady state measurement of radiation detection.

10. The method as claimed in claim 1, wherein said step of measuring comprises measuring said change in the presence of said substance in both said limbs.

11. The method as claimed in claim 1, wherein said presence is measured and recorded as a function of time at a plurality of points in time.

12. The method as claimed in claim 4, wherein said limb is a forearm, and step of measuring comprises providing a gamma camera with its imaging surface ergonomically positioned with respect to a patient, and placing said forearm on said imaging surface of said gamma camera.

13. The method as claimed in claim 12, wherein two said forearms are substantially extended and placed palms down on said imaging surface in order to detect said region of interest of each one of said forearms.

14. The method as claimed in claim 4, further comprising measuring an activity of said bolus prior to injection to establish a reference activity level.

15. The method as claimed in claim 1, wherein said step of measuring comprises:
detecting a first level of said substance at a time of said releasing;
detecting at least one second level of said substance after said releasing;
wherein said determining uses said first level and said at least one second level.

16. The method as claimed in claim 15, wherein said at least one second level is detected at a plurality of predetermined points in time following said releasing.

17. The method as claimed in claim 15, wherein said determining comprises determining a maximum rate of change in said substance from said at least one second level.

18. The method as claimed in claim 17, wherein said determining further comprises recording a time of said maximum rate of change with respect to said releasing.

19. The method as claimed in claim 15, further comprising detecting a base level of said substance prior to blocking, wherein said determining comprises recording a quasi steady state value of said second level for comparison with said base level.

20. The method as claimed in claim 1, wherein said blocking of blood flow comprises occluding one arm so as to raise systolic blood pressure by a predetermined amount.

21. The method as claimed in claim 20, wherein said blood pressure is raised by about 50 mm Hg for approximately 5 minutes.

22. An apparatus for diagnosis of endothelial dysfunction, the apparatus comprising:
a device for measuring a change in a presence of a substance in one limb as a result of a return of blood flow in said one limb, said device outputting a first signal; and
a processor for generating from said first signal data indicative of endothelial dysfunction.

23. The apparatus as claimed in claim 22, further comprising a device detecting a blocking of blood flow in said one limb and a timer for recording said blocking and a releasing of said blocking, said timer providing a second signal fed to said processor.

24. The apparatus as claimed in claim 22, further comprising a device for measuring blood pressure and outputting a blood pressure signal to said processor.

25. The apparatus as claimed in claim 22, wherein said device measures the presence of a radioactive tracer.

26. The apparatus as claimed in claim 22, wherein said device measures an optical property of said substance transcutaneously.

27. The apparatus as claimed in claim 22, wherein said device measures trace amounts of transdermally diffusible molecules.

28. The apparatus as claimed in claim 27, wherein said device comprises a cell sealable against the skin and an analytical instrument for detecting and quantifying selected metabolic marker gases.

29. The apparatus as claimed in claim 22, wherein said device measures temperature of said limb, said temperature being indicative of blood flow into said limb.

30. The apparatus as claimed in claim 29, wherein said device also measures temperature of a contra-lateral limb.

31. The apparatus as claimed in claim 22, wherein said processor measures and records said presence as a function of time at a plurality of points in time.

32. The apparatus as claimed in claim 22, wherein said processor detects a first level of said substance at a time of releasing blood flow blockage, and detects at least one second level of said substance after said releasing, wherein said processor uses said first level and said at least one second level.

33. The apparatus as claimed in claim 32, wherein said at least one second level is detected and recorded at one or more predetermined points in time following said releasing.

34. The apparatus as claimed in claim 33, wherein said processor determines a maximum rate of change in said substance from said at least one second level.

35. The apparatus as claimed in claim 34, wherein said processor records a time of said maximum rate of change with respect to said releasing.

36. The apparatus as claimed in claim 32, further comprising detecting a base level of said substance prior to blocking, wherein said determining comprises recording a quasi steady state value of said second level for comparison with said base level.
